(19) United States
(12) Patent Application Publication
(10) Pub. No.: US 2015/0366957 A1
(43) Pub. Date: Dec. 24, 2015

(54) Melanoma-Associated MHC Class I Associated Oligopeptides and the Uses Thereof

(71) Applicant: Johannes Gutenberg-Universität Mainz, Mainz (DE)

(72) Inventors: Daniela EBERTS, Finthen (DE); Martina FATHO, Worrstadt (DE); Volker LENNERZ, Ober-Olm (DE); Chris SCHMIDT, Herston (AU); Pierre VAN DER BRUGGEN, Brussels (BE); Catherine WÖLFEL, Mainz (DE); Thomas WÖLFEL, Mainz (DE)

(21) Appl. No.: 14/792,919

(22) Filed: Jul. 7, 2015

Related U.S. Application Data
(63) Continuation of application No. 11/991,339, filed on Dec. 30, 2008, now abandoned.

(30) Foreign Application Priority Data
Sep. 1, 2005 (DE) 10 2005 041 616.0

(51) Int. Cl. A61K 39/00; C07K 16/30; A61K 38/17

(52) U.S. Cl. CPC A61K 39/0011; A61K 38/1774; C07K 16/3053; A61K 2039/57; A61K 2039/572; A61K 2039/585

(57) ABSTRACT

The present invention relates to certain melanoma-associated oligopeptides that are recognized by CD8-positive cytotoxic T-lymphocytes (CTLs) as peptide antigen and which elicit a CTL-induced lysis and/or apoptosis of tumor cells. The present invention also relates to the use of these melanoma-associated oligopeptides in cancer therapy.

### CROSS REFERENCE TO RELATED APPLICATIONS

This application is a continuation application of co-pending application Ser. No. 11/991,339, filed Mar. 3, 2008; which is a National Stage Application of International Application Number PCT/EP2006/008533, filed Aug. 31, 2006, which claims priority to German Patent Application No. 10 2005 041 616.0, filed Sep. 1, 2005.

The Sequence Listing for this application is labeled “SeqList-27May14-ST25.txt”, which was created on May 27, 2014, and is 5 KB. The entire content is incorporated herein by reference in its entirety.

### FIELD OF THE INVENTION

The present invention relates to certain melanoma-associated oligopeptides that are recognized by CD8-positive cytotoxic T-lymphocytes (CTLs) as peptide antigen and that elicit a CTL-induced lysis and/or apoptosis of tumor cells. The present invention also relates to the use of these melanoma-associated oligopeptides in cancer therapy.

### BACKGROUND OF THE INVENTION

CD8-positive CTLs are effector cells of the cellular immune system. Their function consists in the specific elimination of infected or degenerated endogenous cells. Among other things the CTLs recognize tumor-specific or tumor-associated peptide antigens (TAAs) that are bound to the class I major histocompatibility complex (MHC) molecules and are present on the surface of the degenerated cells. The recognition of the peptide antigens in the context of MHC class I molecules takes place by specific membrane-bound T-cell receptors (TCR) of the CLTs. After recognition the cell involved is killed in that the CTLs lyse the target cells and/or induce programmed cell death (apoptosis) of the target cells or release cytokines. The present invention relates to those tumor-associated peptides that are capable of binding to a molecule of the human major histocompatibility complex (MHC) class I. Such peptides are used, for example, in the immune therapy of tumor diseases.

In eliminating tumor cells through the immune system the recognition of tumor-associated antigens (TAAs) by components of the immune system plays an outstanding role. This mechanism is based on the precondition that there are qualitative or quantitative differences between tumor cells and normal cells. In order to elicit an anti-tumor response, the tumor cells must express antigens against which an immunological response takes place that is sufficient for eliminating the tumor.

In order to trigger such an immune reaction by cytotoxic T-cells, foreign proteins/peptides must be presented to the T-cells. T-cells only recognize antigens as peptide fragments if these are presented on the cell surfaces by MHC molecules. These MHC molecules (“major histocompatibility complex”) are peptide receptors that normally bind peptides within the cell in order to transport them to the cell surface. This complex of peptide and MHC molecule can be recognized by the T-cells. Human MHC molecules are also designated as human leukocyte antigens (HLA).

There are two classes of MHC molecules: MHC class I molecules, that are found on most cells with a nucleus, present peptides that are formed by the proteolytic degradation of endogenous proteins. MCH class II-molecules are only found on professional antigen-presenting cells (APCs) and present peptides of exogenous proteins that during endocytosis are taken up and processed by APCs. Complexes of peptide and MHC class I are recognized by CD8-positive cytotoxic T-lymphocytes, complexes of peptide and MHC class II are recognized by CD4 helper T-cells.

For a peptide to be able to trigger a cellular immune response it must bind to an MHC molecule. This process is dependent on the allele of the MHC molecule and the amino acid sequence of the peptide. MHC class I-binding peptides normally have a length of 8-12 residues and contain two conserved residues (“anchors”) in their sequence, which interact with the corresponding binding groove of the MHC molecule.

For the immune system to be able to initiate an effective CTL response against tumor-derived peptides, the peptides must not only be able to bind to the particular MHC class I molecules being expressed by the tumor cells, but must also be recognized by T-cells with specific T-cell receptors (TCR, “T-cell receptor”).

The main objective for developing a tumor vaccine is the identification and characterisation of tumor-associated antigens that are recognized by CD8⁺CTLs.

The antigens, recognized by the tumor-specific cytotoxic T-lymphocytes, or their epitopes, respectively, can be molecules from all classes of proteins, such as enzymes, receptors, transcription factors etc. Another important class of tumor-associated antigens are tissue-specific structures, such as, for example, CT (“cancer testis”) antigens that are expressed in various types of tumors and in healthy testicular tissue.

The tumor-associated peptide antigens presented in the context of MHC class I molecules on the surface of tumor cells include, for example, those described in WO 95/25739 A1 for MAGE-3; in U.S. Pat. No. 6,660,276 for melanomas and tyrosinase and in EP 0 668 350 B1 for melanomas and gp100.

TAAs thus represent a starting point for the development of a tumor vaccine. The methods of identifying and characterising the TAAs are on the one hand based on the use of CTLs already induced in patients or are based on generating differential transcription profiles between tumors and normal tissues.

Vaccination studies to date have been limited to the use of a few tumor-associated antigens from the categories of cancer types/germ line and melanosomal proteins. Antigens that are frequently expressed in tumor tissue and are presented in the HLA-alleles frequently occurring in the population were predominantly used. It was also not investigated whether the patients treated in the studies could develop an immune response against the vaccine antigens (see for example Rosenberg, Yang and Restifo Nat Med 10:909, 2004).

However, this strategy does not take into account the presumed fundamental individuality of the interaction between tumor tissue and the body's own immune system. In the overall population during the course of selection processes in transmittable and life-threatening infections, dominant antigens and preferential HLA restriction molecules for immune responses become recognizable, which make an essential contribution to overcome the infection and to subsequent durable protection. Such constant and recurrently observable reaction patterns are not anticipated in interaction with malignant tumors. The reasons for this are:
the inter-individually variable antigen phenotype of malignant tumors (even of the same histological origin) due to genetic and epigenetic events in the tumor,
the pronounced polymorphism of HLA-alleles and
the ability of the individual T-cell repertoire to react to certain epitopes.

Several dozen tumor peptide antigens are known that can be recognized by T-cells. However they are certainly insufficient to explain the anti-tumor T-cell response in patients. In the models we examined, a maximum of 11% of the anti-tumor T-cell clones were directed against known peptides. Instead, they recognized individually specifically mutated peptides or hitherto unknown peptides of structurally normal tumor-associated antigens presented via the patients' own HLA-alleles. This proves the highly individual composition of the anti-tumor T-cell repertoire in humans.

### BRIEF SUMMARY

With respect to this background it is the objective of this invention to provide novel amino acid sequences of immunogenic peptides that are able to bind to molecules of the human major histocompatibility complex (MHC) class I.

### BRIEF DESCRIPTION OF THE DRAWINGS

FIG. 1 schematically shows the course of the vaccinations, the time points of blood withdrawal and the origin of the tumor-reactive MLTCs and T-cell clones in patient model D05-GS.

FIG. 2 shows expansion of tumour-reactive T-cells via autologous MLTC.

FIGS. 3A-3B shows the testing of MLTCs 3.2 in patient model D05-GS (3A) as well as the MLTCs 4.1 from patient model D14-SJR (3B).

FIG. 4 shows the identification of the MAGE-A4/HLA-Cw5 peptide from patient model D05-GS.

FIG. 5 summarizes the recognition of all antigens from patient model D05-GS in chromium release tests.

FIGS. 6A and 6B summarize the recognition of the antigens from the patient model D14-SJR in IFN-γ ELISPOT assays.

### DETAILED DESCRIPTION

In a first aspect this objective is solved in accordance with the invention by a melanoma-specific immunogen, in particular in isolated form, which is a peptide with a length of 9 to about 15 residues and which comprises amino acid sequences selected from the SEQ ID NOs. 1 to 12, more particularly NOs. 2, 3 and 6 to 12, wherein the immunogen elicits a melanoma-specific HLA-restricted CTL response and provided that the peptide does not correspond to the full length sequence of the underlying tumor antigen.

Within the scope of this invention in two melanoma patients (designated as D05-GS and D14-SJR) twelve novel epitopes (in immunogenic peptides) were identified from already known tumor antigens of the categories cancer/germline (=CG) and melanosomal proteins, wherein said epitopes are recognized by tumor-reactive CD8′-T-cells from the patients' blood on their own tumor cells and in association with the HLA class I alleles present in said patients. Two mutated antigens were found encoded by genes with tumor-specific “missense” mutations. As a result of the mutation immunogenic peptides are produced, which are recognized by the T-cells of the respective patient. In the following Table 1 the peptides are listed, which are recognized by T-cells in the lowest concentration, as well as the HLA-alleles from which they are presented.

Within the scope of this invention the terms “immunogen”, “immunogenic oligopeptides”, “oligopeptides” and “peptides” are used interchangeably.

The immunogens identified in accordance with this invention comprise the peptide sequences WQYFFPVIF (SEQ ID NO. 1; HLA-Cw*020202-restricted); KRCFPVIFGK (SEQ ID NO. 2; HLA-B*270502-restricted); KVDELAHFL (SEQ ID NO. 3; HLA-Cw*0501-restricted); NMVPFFPPV (SEQ ID NO. 4; HLA-A*020101-restricted); MPREDAHFIY (SEQ ID NO. 5; HLA-B*510101-restricted); LYPEWTEAQR (SEQ ID NO. 6; HLA-A*24020101-restricted); KYKDYFPVI (SEQ ID NO. 7; HLA-Cw*0702 or HLA-A*24020101-restricted); ASSASSTLYL (SEQ ID NO. 8; HLA-Cw*150201-restricted), SSASSTLYL (SEQ ID NO. 9; HLA-Cw*150201-restricted) and VPSGVIPNL (SEQ ID NO. 13; HLA-B*070201-restricted). In accordance with the invention also two TAA-epitopes mutated when compared to the wild-type sequence were identified (mutated amino acids underlined), namely LRTKVYAEL (SEQ ID NO. 10; HLA-B*270502-restricted) and YPPPPPALL (SEQ ID NO. 11; HLA-B*510101-restricted).

With the aid of the immunogens/oligopeptides in accordance with the invention cytotoxic T-cells can be generated, which develop an antigen-specific MHC-restricted cytotoxic activity against the tumor cells, expressing immunogens/oligopeptides of the invention and destroy them.

Therefore, these peptides open up the possibility of an effective tumor therapy in which the suppression of an immune reaction which is often observed in tumor patients can be reversed.

As can be seen in the embodiments, the inventors have succeeded in proving that against the oligopeptides according to the invention cytotoxic T-cells can be produced very effectively, which in an MHC-class I restricting manner kill the tumor cells, that produce corresponding TAAs and carry them on their surface. Via MHC class I-molecules these tumor cells present fragments of all proteins produced by them. If cytotoxic T-cells now recognize the peptide presented by an MHC class I-molecule, through which they were originally activated, they kill these cells. Thus, the peptide, presented by MHC class I-molecules of TAA-expressed cells offers the advantageous possibility of specifically eliciting an immune reaction against tumors that produce the corresponding TAAs.

In accordance with the invention it is preferred that the immugens in accordance with the invention have a length of 9 to 11 residues. N and/or C teiininal of the “core region” of SEQ ID NOs. 1 to 12 further amino acids can be present. These further amino acids are preferably selected from the original sequence of the corresponding TAA but can also be selected from the other 20 known amino acids. Accordingly, preferred peptides have a lenght of 500, 400, 300, 200, 100 or 50 amino acids, without however corresponding to the entire sequence of the corresponding TAA (also e.g. MAGE, gp100, etc., see Table 1). These extended peptides are in particular suited for the external loading of cells and are (without intending to be bound to this theory) processed further to a presentation. More preferred are peptides with a length of 9, 10, 11, 12, 13, 14, 15, 16, 17, 18, 19 or 20 amino acids. Such extended immunogens are also comprised by the term “oligopeptides” in accordance with the invention.

The invention also relates to a fusion protein composed of one of the aforementioned oligopeptides and of another peptide or protein and which is suitable for use as a diagnostic or therapeutic or phrophylactic agent or generally for a detection and/or manipulation of T-cells that recognize one of the oligopeptides shown in SEQ ID NOs. 1 to 12. For example, fusion proteins are possible that consist of a carrier protein such as, for example, HSA, collagen or other proteins and one or more of the oligopeptides of the invention. These fusions can also contain one or more of the epitopes according to the invention as amino acid cassettes. The polynucleotides coding for this fusion protein are also the subject matter of the present invention.

Modified forms of the peptides of SEQ ID NOs. 1 to 12 can also result in the desired immune response.

In terms of the invention, “modified” means any chemical, enzymatic or other modification of the peptide. This can, for example, already take place during the production of the peptide or later on by the removal or addition of individual amino acid residues, the substitution of individual amino acid residues or also by chemical modification of individual amino acid residues via the attachment of certain chemical groups.

A further aspect of the invention relates to an immunogen according to the invention, wherein the immunogen has an amino acid sequence derivable by amino acid substitution, deletion, insertion, addition, inversion and/or by chemical or physical modification of one or more amino acids thereof, wherein said amino acid sequence represents a functional equivalent to the amino acid sequence of one of the peptides of SEQ ID NOs. 1 to 12, and wherein said immunogen is an epitope for CD8-positive CTLs and is suitable for inducing an immune response of CD8-positive CTLs against tumor cells, wherein said immune response is restricted to human leukocyte-antigen of molecule group MHC class I, allele variant A or B. A further aspect of the invention relates to a retro-inverse peptide or pseudopeptide, characterized in that it corresponds to an immunogen according to any one of claims 1 to 4 wherein —NH—CO-bonds or other non-peptide bonds are formed instead of the —CO—NH-peptide bonds (cf. e.g. Meziere C, Viguier M, Dumortier H, Lo-Man R, Leclerc C, Guillet J G, Briand J P, Muller S. In vivo T helper cell response to retro-inverse peptidomimetics. J Immunol. 1997 Oct. 1; 159(7):3230-7.).

Particularly preferred is an immunogen in accordance with the invention wherein the immunogen is identical to one of the peptides of the SEQ ID NOs. 1 to 12.

The invention also relates to a fusion protein which preferably consists of one of the above-described oligopeptides, a heavy chain of the HLA molecule and a flexible linker and is designed in such a way that the oligopeptide is configured (suitable or able or enabled, respectively) to occupy the peptide binding groove of the HLA molecule and wherein the fusion protein is suitable for use as a diagnostic, therapeutic or prophylactic agent or generally for a detection and/or manipulation of T-cells that recognize one of the oligopeptides set out in the SEQ ID NOs. 1 to 12. The polynucleotides coding for this fusion protein are also the subject matter of the present invention.

In a further preferred embodiment the invention relates to a polynucleotide comprising a nucleotide sequence, which comprises a sequence segment that codes for at least one immunogen in accordance with the invention. In addition to that, the nucleic acid optionally has further sequences which are necessary for expressing the nucleic acid sequence corresponding to the peptide. The nucleic acid used can be contained in a vector suitable for allowing expression of the nucleic acid sequence corresponding to the peptide in a cell.

Such a nucleic acid has the advantage that it is chemically more stable and less sensitive than the peptides. The handling is therefore easier than that of the peptides and the suitability for storage of nucleic acids is almost infinite. They are chemically and/or molecularbiologically very cost-effective and can in principle be produced in unlimited quantities. Nucleic acid sequences required for expression have, like a vector containing the nucleic acid, the advantage that it is thereby possible to produce large quantities of the peptides very cost-effectively by using cellular expression systems via the nucleic acids.

However, the nucleic acids can also be used to transform antigen-presenting cells, in particular dendritic cells, in such a way that they themselves produce the corresponding peptides and then by way of MHC I-molecules present them to cytotoxic T-cells or their precursor cells. The advantage of this is that the presentation of the corresponding peptides by the antigen-presenting cells takes place over a longer term than the presentation of peptides only provided from outside.

A further aspect of the present invention is a pharmaceutical composition for the in vivo or in vitro activation of T-cells, in particular CD8-positive cytotoxic T-lymphocytes, that comprise at least one immunogen in accordance with the invention and/or at least one retro-inverse peptide or pseudopeptide in accordance with the invention and/or at least one fusion protein in accordance with the invention and/or at least one polynucleotide in accordance with the invention, optionally with acceptable carriers and excipients.

Yet a further aspect of the present invention is a recombinant DNA or RNA vector molecule that contains at least one or more polynucleotide(s) in accordance with the invention and that can be expressed in cells of autologous, allogenic, xenogenic or microbiological origin. A further aspect of the present invention is a respective host cell, which contains such a polynucleotide or such a vector molecule.

A further aspect of the invention relates to a pharmaceutical composition that contains a peptide in accordance with the invention or a nucleic acid in accordance with the invention in an amount that effectively elicits an MHC I-restricted immune response.

For this the peptides and/or nucleic acids can be prepared in the appropriate usual galenicals. In the case of peptides this could, for example, be preparations usually used for vaccinations and containing an adjuvant. In the case of nucleic acids, a preparation with liposomes or vesicles is also possible. By using an appropriate pharmaceutical preparation it is possible to treat organisms directly without having to extract antigen-presenting cells or their precursor cells beforehand in order to initially cultivate them and administer them to the patients after treatment with peptides or nucleic acids. By way of appropriate pharmaceutical preparation tumor treatment can take place in the form of an inoculation.

A further aspect of the invention relates to the use of a nucleic acid in accordance with the invention in the context of gene therapy.

Here, gene therapy can take place in the form of the above-described transformation of antigen-presenting cells, in particular dendritic cells, which, or the precursor cells thereof have previously been taken from the body of an organism to be treated, in order to be re-administered into the body after transformation. As has already been mentioned, compared to the use of peptides the longer presentation period is advantageous.

Another possibility consists in introducing the nucleic acid into the body in such a way that it is selectively taken up and expressed by antigen-presenting cells, in particular dendritic cells. This application has the advantage that apart from the administration no further measures are necessary, such as cultivation and selective reproduction of extracted dendritic cells or their precursor cells.

The invention also relates to the use of a nucleic acid in accordance with the invention for the in vitro transformation or transfection of cells. The in vitro use of the nucleic acid has the advantage that processes, such as, for example, electroporation, and/or auxiliary substances, such as calcium phosphate or DEAE dextrane, can be used which considerably facilitate and improve the uptake of nucleic acids in cells but cannot be employed in vivo.

As has already been mentioned, antigen-presenting cells, in particular dendritic cells, can be treated which for example have been taken from their precursor cells from a patient and have subsequently been re-introduced into the patient.

Another aspect of the present invention is the use of at least one peptide in accordance with the invention or a nucleic acid in accordance with the invention for eliciting an immune reaction in connection with a tumor therapy or a treatment for preventing a tumor.

Advantageous here is the fact that the frequently observed immunosuppression and tolerance to TAA in a tumor disease can be reversed by the use of peptides and nucleic acids in accordance with the invention. The use in accordance with the invention can also be employed in addition to established tumor therapies.

A preventative treatment is of benefit possibly mainly to persons who are at increased risk of developing a tumor, because, for example, they are heriditary predisposed or because they have already had a tumor before.

In a further development, at least one of the peptides or nucleic acids in accordance with the invention is incubated together with antigen-presenting cells, in particular dendritic cells, and only then introduced into an organism from which the antigen-presenting cells or their precursors were previously extracted.

The advantage of this method is that the success when eliciting an immune reaction this way is more ensured and controllable compared to injecting a peptide together with an adjuvant wherein the immune reaction is sometimes stronger and sometimes weaker. However, particularly in case of a tumor therapy, one should always be able to ensure success when eliciting an immune reaction so as not to lose valuable time due to ineffective treatment.

The oligopeptides in accordance with the invention (SEQ ID NOs. 1 to 12 and their derivatives) can be used for the active and passive immunization of patients with melanomas in whom the corresponding epitopes of SEQ ID NOs. 1 to 12 are presented in order to effect the induction, generation and expansion of specific cytotoxic T-lymphocytes that are able to specifically kill the tumor cells of the respective patients and thereby mediate and/or support any healing.

The derivatives of the oligopeptides of SEQ ID NOs. 1 to 12 and also the retro-inverse peptides or pseudopeptides derived therefrom have the advantage compared to the relevant original oligopeptide itself that a potential functional self-tolerance (vis-à-vis the oligopeptides of SEQ ID NOs 1 to 12) can be avoided at T-cell level. Whereas the oligopeptide due to the (low) expression in some normal tissues is, in some circumstances, a so-called tolerogen in the relevant organism (patient's body) and is not immunogenic for the organism's own (patient's own) CTLs, the derivatives of these oligopeptides (SEQ ID NOs.: 1 to 12) are generally recognized as antigens and induce the activation and expansion of CTLs. These derivative-induced CTLs generally have a high cross-reactivity with the relevant wild-type sequence SEQ ID NOs. 1 to 12 and as a result also induce the lysis and/or apoptosis of such (tumor) cells, which present the SEQ ID NOs. 1 to 12 in the context of HLA on their surface.

Particularly preferred derivatives of the oligopeptides of SEQ ID NOs. 1 to 12 are those that naturally occur in other mammals or vertebrates, for example homologues of SEQ ID NOs. 1 to 12 from mice. The (protein) and peptide homologues of SEQ ID NOs. 1 to 12 and nucleic acids encoding them can be relatively easily obtained from the organism, namely directly and with conventional isolation methods.

The oligopeptides SEQ ID NOs. 1 to 12 and their derivatives as well as the retro-inverse peptides or pseudopeptides can be produced by way of conventional peptide synthesis methods and the nucleotide sequences encoding these oligopeptides can be obtained with known chemical and molecularbiological methods.

It is also possible to produce a fusion protein with the above oligopeptides in accordance with the invention, a flexible linker and a heavy chain of the HLA molecule, namely in such a way that the oligopeptide is enabled (in a position to or suitable, respectively) to occupy the peptide binding groove of the HLA molecule. These fusion proteins and the polynucleotides encoding them are particularly suitable as (active substances of) a diagnostic, therapeutic or prophylactic agent or in general for the detection and/or manipulation of T-cells that recognize one of the oligopeptides set out in SEQ ID NO. 1 to 12.

The oligopeptides in accordance with the invention (SEQ ID NOs. 1 to 12 and their derivatives) as well as the retro-inverse peptides or pseudopetides and the above-described fusion proteins are suitable for both in vivo induction of T-lymphocytes in the patient as well as for in vitro induction and expansion of respective reactive T-lymphocytes inherent or alien to the patient.

For an in vivo induction and expansion of T-lymphocytes in the patient various methods can be considered, for example (a) the injection of the oligopeptides in accordance with the invention and/or one or more derivatives of one or both of these oligopeptides and/or a retro-inverse peptide or pseudopeptide and/or a fusion product as described above as pure peptide or together with adjuvants or with cytokines or in a suitable release system, such as liposomes, (b) the injection of one or more of at least the oligopeptides in accordance with the invention or their derivatives and/or for one of the retro-inverse peptides or pseudopeptides and/or for nucleic acids encoding one of the fusion proteins—in pure or complex form or in the form of viral or non-viral vectors, or together with release systems, such as cationic lipids or cationic polymers, (c) the loading of cells of autologous, allogenic, xenogenic or microbiological origin with the oligopeptides or their derivatives in accordance with the invention or retro-inverse peptides or pseudopeptides analog thereto, (d) the loading of cells of autologous, allogenic, xenogenic or microbiological origin with oligopeptides in accordance with the invention or homologues of other species, so that as a result the oligopeptide and/or derivatives of one or both of these oligopeptides are presented on the relevant cells, or (e) the transfection or infection of cells of autologous, allogenic, xenogenic or microbiological origin with nucleic acids (again in “pure” or complexed form or in form of viral or non-viral vectors) coding for at least the oligopeptide in accordance with the invention or their derivatives or a for a retro-reverse peptide or pseudopeptide derived therefrom or a for an above-described fusion protein.

In the case of in vitro induction and expansion the T-lymphocytes obtained in vitro are subsequently administered to the patient by way of infusion or injection or similar processes.

The invention therefore also relates to the use of the oligopetides in accordance with the invention and/or their derivatives and/or retro-inverse peptides or pseudopeptides analog thereto and/or the above-described fusion proteins and/or at least one polynucleotides that codes at least for the oligopeptide in accordance with the invention and/or a derivative of one or both of the oligopeptides, for the production of diagnostic agents, in particular MHC-tetramers or MHC-dimers or other structures to which at least one such oligopeptide or retro-inverse peptide or pseudopeptide in accordance with the invention is associated by way of covalent or non-covalent binding and/or prophylactic and/or therapeutic agents (in particular vaccines) for detecting and/or influencing and/or generating and/or expanding and/or controlling the activation and functional status of T-cells, in particular CD8-positive CTLs.

As therapeutic and/or prophylactic agents, in particular vaccines or injections of infusion solutions are considered, which as the active agent(s) (a) contain the oligopeptide in accordance with the invention and/or at least one derivative of one of these oligopeptides and/or at least one retro-inverse peptide or pseudopeptide analog to one of these oligopeptides or their derivatives and/or at least one of the fusion products described above and/or which (b) contain a nucleic acid that codes for the oligopetide in accordance with the invention or at least for a derivative of one of these oligopeptides, and/or (c) contain the T-lymphocytes produced in vitro which are specifically directed against the oligopeptide and/or derivative(s) thereof and/or against a retro-inverse peptide or pseudopeptide analog to one of these oligopeptides or a derivative of these oligopeptides.

Recombinant DNA or RNA vector molecules containing one or more polynucleotide(s) that code for at least one oligopeptide of SEQ ID NOs. 1 to 12 and/or for at least one derivative of one of these oligopeptides and which can be transcribed or expressed, respectively, in cells of autologous, allogenic, xenogenic or microbiological origin are particularly suitable for producing the diagnostic or also the therapeutic or also the prophylactic agents. The invention therefore also comprises recombinant DNA or RNA vector molecules and host cells that contain these vector molecules.

In accordance with the invention, as diagnostic or therapeutic or prophylactic agents or generally for the detection and/or manipulation of respective cells overexpressing TAA, polyclonal, monoclonal or recombinant antibodies can be used, which are directed against the oligopeptides of SEQ ID NOs. 1 to 12 and/or against a derivative of one of these oligopeptides and/or against a retro-inverse peptide or pseudopeptide analog to one of these oligopeptides or their derivatives and/or against a fusion product described above or which react with a complex of one of the respective oligopeptides and/or their derivatives and/or retro-inverse peptide(s) and/or pseudopeptide(s) thereof, respectively, and HLA

The use of the oligopeptide of SEQ ID NOs. 1 to 12 and/or a derivative of these oligopeptides and/or a retro-inverse peptide or pseudopeptide analog to one of these oligopeptides or to a derivative of these oligopeptides or a fusion protein for producing polyclonal, monoclonal or recombinant antibodies against such a oligopeptide in accordance with the invention or retro-inverse peptide or pseudopeptide, relatively, and the respective antibody itself/antibodies themselves also form part of the present invention.

In accordance with the invention, as diagnostic or therapeutic or prophylactic agents or generally for the detection and/or manipulation of respective cells overexpressing TAA, polyclonal, monoclonal or recombinant HLA-restricted T-cell receptors or functionally equivalent molecules thereof can be used that are specific for an oligopeptide of SEQ ID NOs. 1 to 12 and/or a derivative of one of these oligopeptides and/or for retro-inverse peptides or pseudopeptides analog thereto and/or for an fusion product described above. The T-cell receptors or functionally equivalent molecules thereof can be of autologous, allogenic or xenogenic origin.

Consequently the following also form part of the present invention: the use of an oligopeptide of SEQ ID NOs. 1 to 12 and/or a derivative of one of these oligopeptides and/or retro-inverse peptides or pseudopeptides analog thereto or the use of polynucleotides with a nucleotide sequence that codes for at least one oligopeptide SEQ ID NOs. 1 to 11 and/or a derivative of these oligopeptides for the production of polyclonal, monoclonal or recombinant HLA-restricted T-cell receptors or functionally equivalent molecules thereof with specificity for such an oligopeptide or retro-inverse peptide or pseudopeptide in accordance with the invention, the relevant T-cell receptor(s) itself and functionally equivalent molecules thereof, as well as polynucleotides that code for these T-cell receptors or functionally equivalent molecules thereof, expression vectors with the ability to express these T-cell receptors or functionally equivalent molecules thereof as well as corresponding host cells, as above.

The invention also relates to reagents for the in vivo or in vitro activation of T-cells, in particular CD8-positive CTLs, characterized in that they are produced by using a oligopeptide of SEQ ID NOs. 1 to 12 and/or at least one derivative of one of these oligopeptides and/or at least one retro-inverse peptide or pseudopeptide analog thereto or at least one fusion protein described above and/or by using at least one polynucleotide that codes at least for the oligopeptide or its derivative(s) and/or by using the corresponding TAA-protein or homologues thereof of other species. These reagents can, in particular, be therapeutic agents, above all vaccines.

A further aspect of the present invention is a medicinal product for the treatment of diseases which are associated with the immunogens of SEQ ID NOs. 1 to 12 according to the invention, in particular melanomas, characterized in that the medicinal product contains at least one immunogen and/or at lest one retro-inverse peptide or pseudopeptide and/or at least one fusion protein and/or at least one polynucleotide and/or at least one T-cell receptor and/or at least one vector module and/or at least one host cell and/or at least one antibody in accordance with the invention, optionally together with suitable additives and excipients.

Then, a further aspect of the present invention relates to the use of an immunogen in accordance with the invention and/or a retro-inverse peptide or pseudopeptide in accordance with the invention and or a fusion protein in accordance with the invention for producing diagnostic and/or therapeutic and/or prophylactic agents for detecting and/or influencing and/or generating and/or expanding and/or controlling the activation and functional status of T-cells, in particular CD8-positive cytotoxic T-lymphocytes.

An even further aspect of the present invention relates to the use of at least one immunogen in accordance with the invention and/or retro-inverse peptide or pseudopeptide in accordance with the invention and/or fusion protein in accordance with the invention for eliciting an immune reaction in connection with a tumor therapy or treatment preventing the formation of a tumor.

Finally, the present invention relates to a method of treating a patient against melanoma, comprising the administration of a therapeutically effective amount of at least one immunogen and/or at least one retro-inverse peptide or pseudopeptide and/or at least one fusion protein and/or at least one polynucleotide and/or at least one T-cell receptor and/or at least one vector molecule and/or at least one host cell and/or at least one antibody of the invention in an amount thereby achieving a therapeutic effect. Another aspect is a method of eliciting a melanoma-specific CTL response comprising the administration of response-eliciting amount of the melanoma-specific immunogen in accordance with the invention, optionally with excipients as set out above.

The provision of additional defined immunogens in accordance with the invention allows therapeutic vaccination studies to be carried out in a higher number of patients. In connection with this the chances of including individually relevant antigens are increased.

It is understood that the aforementioned features and the features described herein below cannot only be used in the respective indicated combination but also in other combinations or alone, without departing from the scope of the present invention.

The invention will now be explained in more detail below with the aid of examples of embodiment and referring to the attached drawings, but without being restricted thereto.

TABLE 1
T-cell recognized antigens in accordance with the
invention in melanoma patients D05-GS and D14-SJR

| Peptide | | Restricting | Antigen | SEQ | | Prevalence | |
|---|---|---|---|---|---|---|---|
| sequence | Gene | HLA-allele | name | ID NO. | Method | (%) | Patient |
| WQYFFPVIF | MAGE-A3,-A6^{LICR} | HLA-Cw*020202 | MAGE-A3,6/Cw2 | 1 | 1 | 9-20 | D05-GS |
| KRCFPVIFGK | MAGE-A4 | HLA-B*270502 | MAGE-A4/B27 | 2 | 1 | 3-4 | D05-GS |
| KVDELAHFL | MAGE-A4 | HLA-Cw*0501 | MAGE-A4/Cw5 | 3 | 2 | 4-8 | D05-GS |
| NMVPFFPPV | HSTRP-2 | HLA-A*020101 | HSTRP2/A2 | 4 | 4 | 41-43 | D05-GS |
| MPREDAHFIY | Melan-A | HLA-B*510101 | Melan-A/B51 | 5 | 1 | 4-37 | D14-SJR |
| LYPEWTEAQR | gp100 | HLA-A*24020101 | gp100/A24 | 6 | 1 | 17-20 | D14-SJR |
| KYKDYFPVI | MAGE-C2 | HLA-A*24020101 | MAGE-C2/A24 | 7 | 1/3 | 8-10/22-26 | D14-SJR |
| | | HLA-Cw*0702 | MAGE-C2/Cw7 | | | | |
| ASSASSTLYL | MAGE-C2 | HLA-Cw*150201 | MAGE-C2/Cw15(1) | 8 | 1 | 3-8 | D14-SJR |
| SSASSTLYL | MAGE-C2 | HLA-Cw*150201 | MAGE-C2/Cw15(1) | 9 | 1 | 3-8 | D14-SJR |
| LRTKVYAEL** | CCT6Amut | HLA-B*270502 | CCT6Amut/B27 | 10 | 4 | Unknown | D05-GS |
| YPPPPPALL** | N-WASPmut | HLA-B*510101 | N-WASPmut/B51 | 11 | 4 | Unknown | D14-SJR |
| YMDGTMSQV* | Tyrosinase | HLA-Cw*0501 | Tyr/Cw5 | 13 | 1 | 14-25 | D05-GS |
| VPSGVIPNL | MAGE-C2 | HLA-B*070201 | MAGE-C2/B7 | 12 | 2 | 12 | D14-SJR |

*: Peptide has already been published in connection with HLA-A2;
**: mutated amino acid underlined;
prevalence: prevalence to be expected in patients with malignant melanomas, determined from the frequency of expression of the relevant antigen in melanomas (according to literature) and the frequency at which the presenting HLA-allele occurs in Northern European and North American Caucasian population groups;
Method:
1: identified by panel testing with MLTC-responders;
2: identified by panel testing with clonal T-cells;
3: identified by cDNA bank screening with MLTC responders;
4: identified by cDNA bank screening with clonal T-cells.

### Examples

The methodological procedure leading to the identification of the melanoma peptide antigens in accordance with the invention is described below. Reference is also made to the attached drawings.

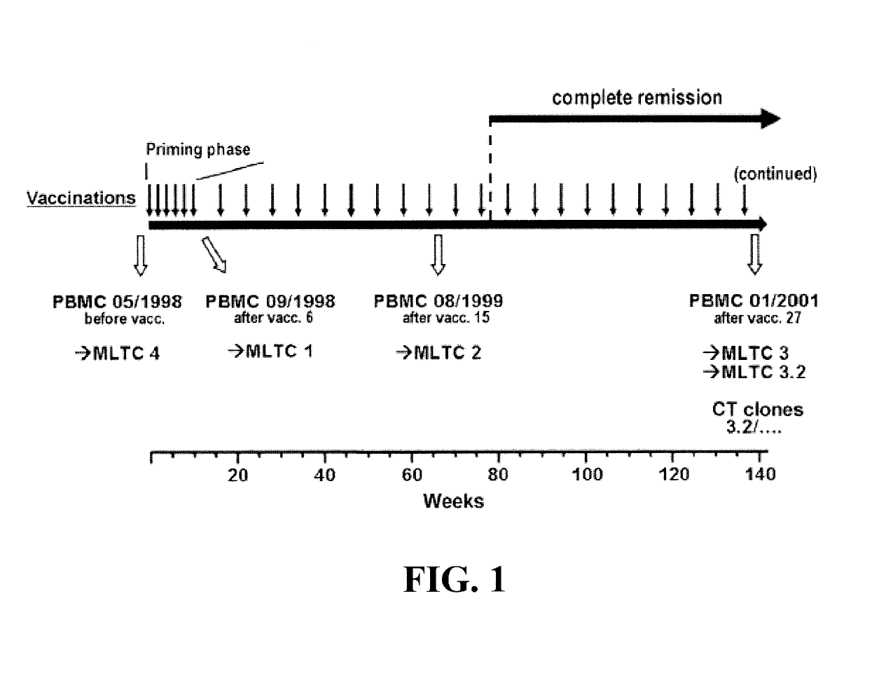

Stably growing melanoma cell lines were established from the tumors of the patients D05-GS and D14-SJR. In addition, over a period of several years blood was taken from these patients who were taking part in a vaccination study in Brisbane/Australia. FIG. 1 schematically shows the course of the vaccinations, the time points of blood withdrawal and the origin of the tumor-reactive MLTCs and T-cell clones in patient model D05-GS. Analog thereto, the corresponding steps were also carried out in patient model D14-SJR (not shown).

From these blood samples lymphocytes were isolated and cryo-conserved in Brisbane. As a contribution to the invention Prof Pierre van der Bruggen of the Ludwig Institute for Cancer Research/Brussels (abbreviated as LICR) provided a panel of cDNA clones in expression plasmides that code for a total of 31 antigens of the cancer/germline (CG) category and the melanocyte-differentiation antigen category (Table 2).

From the melanoma line of patient D05-GS the inventors cloned by means of RT-PCR the HLA-alleles HLA-A*020101, HLA-B*270502, HLA-B*44020101, HLA-Cw*0501, HLA-Cw*020202, and from the melanoma line of patient D14-SJR the HLA-alleles HLA-A*030101, HLA-A*24020101, HLA-B*070201, HLA-B*510101 and HLA-Cw*150201. The allele HLA-Cw*0702 also carried by patient D14-SJR was made available to the inventors in the context of a collaboration. From both melanoma lines cDNA banks were constructed in pcDNA3.1.

TABLE 2
Testing of candidate antigens with tumor-reactive MLTC.

| Patient D05-GS | | | | | | Patient D14-SJR | | | | |
|---|---|---|---|---|---|---|---|---|---|---|
| | | HLA- | | | | | HLA- | HLA- | | |
| | HLA-A* | B* | HLA-B* | HLA-Cw* | HLA-Cw* | HLA-A* | B* | B* | HLA-Cw* | HLA-Cw* |
| Plasmide/Antigen | 02010101 | 270502 | 44020101 | 020202 | 0501 | 24020101 | 070201 | 510101 | 070201 | 0150201 |
| pcDNAI/gp100 | — | — | — | — | — | + | — | — | — | — |
| pcDNAI/tyrosinase | + | — | — | — | + | — | — | — | — | — |
| pcDNAI/MART-1 | + | — | — | — | — | — | — | + | — | — |
| pcDNAI/MAGE-A1 | — | — | — | — | — | — | — | — | — | — |
| pcDNA3/MAGE-A2 | — | — | — | — | — | — | — | — | — | — |
| pcDNAI/MAGE-A3 | — | — | — | + | — | — | — | — | — | — |
| pcDNAI/MAGE-A4 | — | + | — | — | + | — | — | — | — | — |
| pcDNA3/MAGE-A6 | — | — | — | + | — | — | — | — | — | — |
| pcDNAI/MAGE-A8 | — | — | — | — | — | — | — | — | — | — |
| pcDNAI/MAGE-A9 | — | — | — | — | — | — | — | — | — | — |
| pcDNAI/MAGE- | — | — | — | — | — | — | — | — | — | — |
| A10 | | | | | | | | | | |
| pcDNAI/MAGE- | — | — | — | — | — | — | — | — | — | — |
| A11 | | | | | | | | | | |
| pcDNAI/MAGE- | — | — | — | — | + | — | — | — | — | — |
| A12 | | | | | | | | | | |
| pcDNAI/MAGE-B2 | — | — | — | — | — | — | — | — | — | — |
| pcDNAI/MAGE-C1 | — | — | — | — | — | — | — | — | — | — |
| pcDNAI/MAGE-C2 | — | — | — | — | — | + | + | — | + | + |
| pcDNAI/BAGE | — | — | — | — | — | — | — | — | — | — |
| pcDNAI/GAGE-1 | — | — | — | — | — | — | — | — | — | — |
| pcDNAI/GAGE-2 | — | — | — | — | — | — | — | — | — | — |
| pcDNAI/GAGE-3 | — | — | — | — | — | — | — | — | — | — |
| pcDNAI/GAGE-4 | — | — | — | — | — | — | — | — | — | — |
| pcDNAI/GAGE-5 | — | — | — | — | — | — | — | — | — | — |
| pcDNAI/GAGE-6 | — | — | — | — | — | — | — | — | — | — |
| pcDNAI/GAGE-7B | — | — | — | — | — | — | — | — | — | — |
| pcDNAI/GAGE-8 | — | — | — | — | — | — | — | — | — | — |
| pcDNAI/PRAME | — | — | — | — | — | — | — | — | — | — |
| pcDNAI/RAGE-1 | — | — | — | — | — | — | — | — | — | — |
| pcDNAI/RAGE-2 | — | — | — | — | — | — | — | — | — | — |
| pcDNA3/RAGE-3 | — | — | — | — | — | — | — | — | — | — |
| pcDNAI/RAGE-4 | — | — | — | — | — | — | — | — | — | — |
| pcDNA3/NY- | + | — | — | — | — | — | — | — | — | — |
| ESO-I | | | | | | | | | | |

+ denotes recognition by CD8⁺ MLTC or T-cell clones;

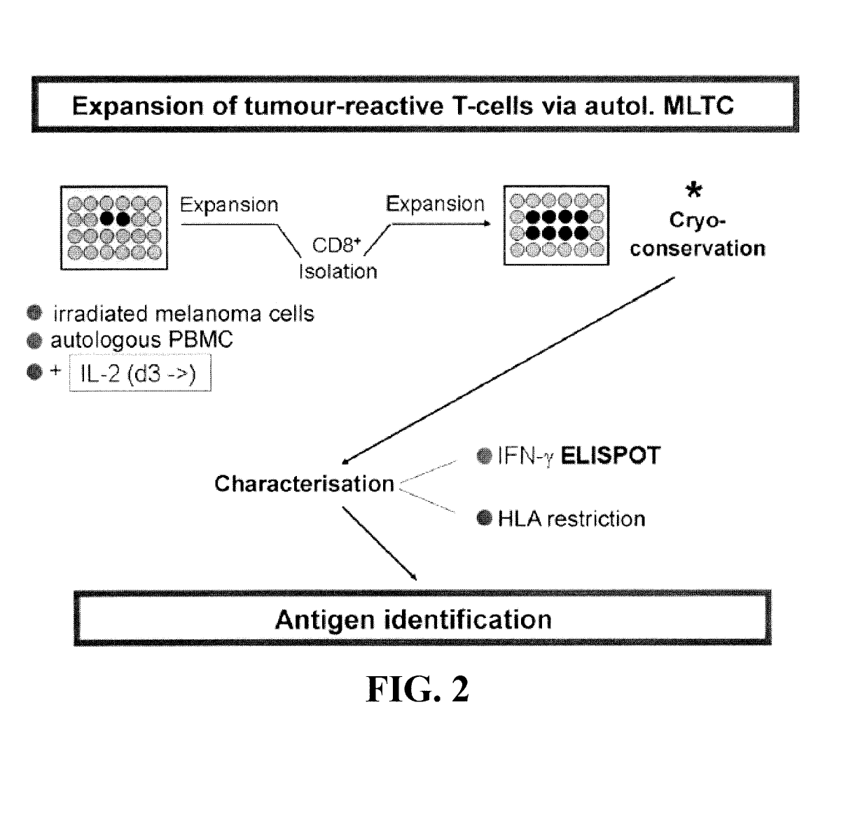

In the so-called mixed lymphocyte/tumor cell cultures (MLTC) blood lymphocytes were stimulated with the respective (autologous) tumor cells originating from the same donor (FIG. 2).

In the responder-lymphocytes tumor-specific T-cells were thereby enriched. In each patient model several such MLTC-responder populations were generated with blood lymphocytes from several years and cryo-conserved at various time points after prior cleaning of CD8-positive T-cells (FIG. 2). Prior to cryo-conservation they were checked for recognition of autologous melanoma cells and autologous EBV-transformed B-cells. In addition, the preferential HLA-restriction of the MLTC-responders was determined by blocking tumor cell recognition with HLA group-specific antibodies (not shown).

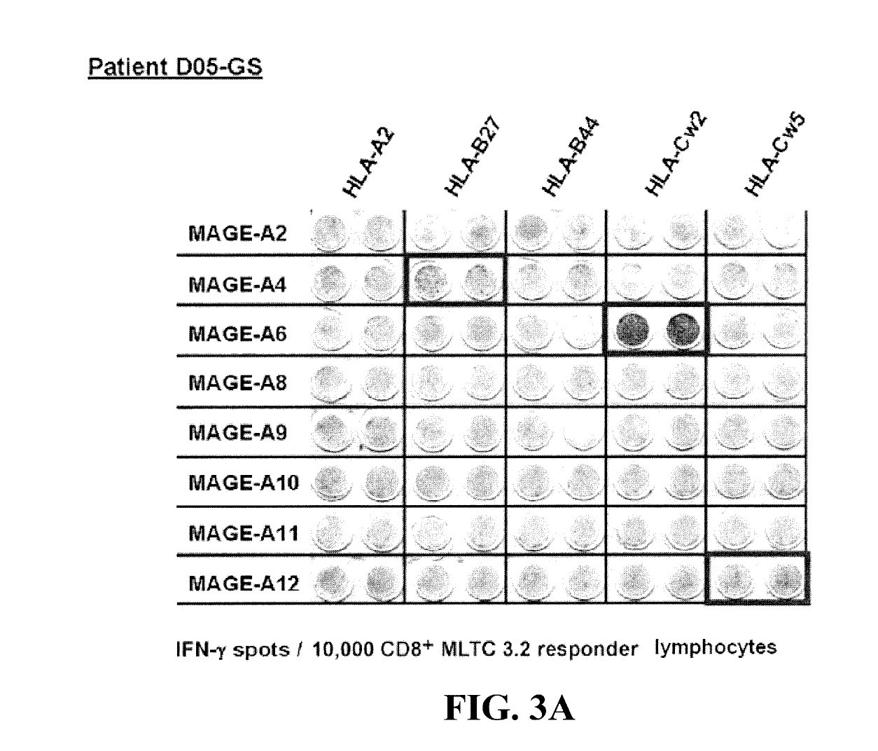

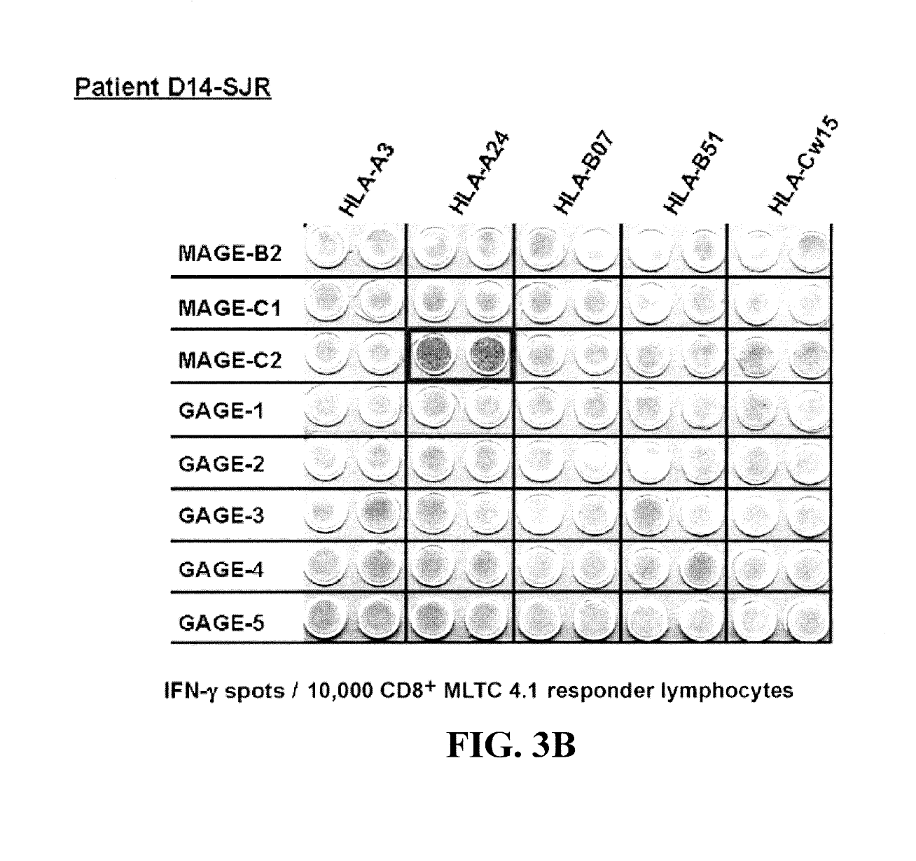

MLTC-populations, cryo-conserved at various time points were checked in a “panel test” of reactivity to known melanoma-associated antigens. For this the aforementioned antigen-coding expression plasmids were transfected with each of the HLA-alleles of both patients in COS-7-cells or 293T-cells (Table 2). The transfectants were then tested in the interferon-gamma ELISPOT assays for recognition by MLTC-responders. FIGS. 3A-3B each show an example of the testing of MLTCs 3.2 in patient model D05-GS (FIG. 3A) as well as the MLTCs 4.1 from patient model D14-SJR (FIG. 3B). Overall, the inventors determined T-cell reactivities against the following, hitherto not known, antigen-HLA-combinations (see also Table 2):

### Patient (Model) D05-GS:

Tyrosinase/HLA-Cw5; MAGE-A3/HLA-Cw2; MAGE-A6/HLA-Cw2; MAGE-A4/HLA-B27 and MAGE-A4/HLA-Cw5

### Patient (Model) D14-SJR:

Melan-A/HLA-B51; gp100/HLA-A24; MAGE-A3/HLA-A24; MAGE-A6/HLA-A24; MAGE-C2/HLA-A24; MAGE-C2/HLA-B7; MAGE-C2/HLA-Cw7 and MAGE-C2/HLA-Cw15

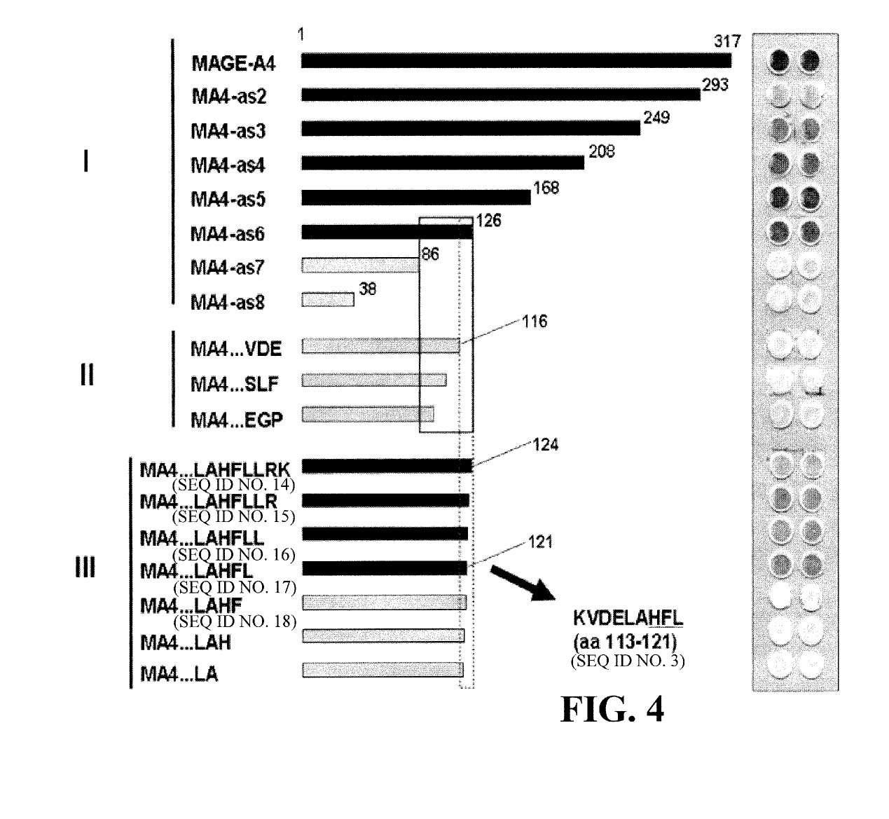

In the next step the peptide-coding gene regions for these antigens were identified. For this antigen-coding cDNAs were fragmented by way of PCR, the fragments were again cloned in an expression vector and cotransfected with the respective presenting HLA alleles in COS-7 or 293 T-cells and tested in the IFN-γ-ELISPOT test for recognition by the MLTC populations or T-cell clones. Recognized fragments were shortened further and tested until the C-terminus of the recognized peptide was identified. FIG. 4 shows, for example, the identification of the MAGE-A4/HLA-Cw5 peptide from patient model D05-GS. The numbers show the length of the polypeptide chains in amino acids which are encoded by the cDNA fragments. The peptide was identified in three steps (I-III). From testing of the first eight fragments it was concluded that the peptide-coding region was between amino acids 86 and 126 (I). The second test showed that the C-terminus of the recognized peptide had to lie between amino acids 116-126 (II). In the concluding testing of fragments that coded for polypeptides that were shortened at the C-terminus successively by one amino acid, the C-terminus of the recognized peptide was determined as amino acid 121 (III). After precise localisation of the C-terminal peptide end peptides of 9 and 10 amino acids in length were synthesized and checked for recognition by the T-cells. Table 1 shows the peptides identified in this way.

The anti-tumor reactivity of the MLTC-responders exceeded the sum of reactivities against known melanoma-associated antigens. MLTC-populations were cloned in the borderline dilution procedure and cytotoxic T-cell clones were selected, which recognized melanoma cells but none of the known antigens.

Such clones were selected for the cDNA bank screening for searching for novel antigens. The cDNA libraries were divided into pools of 100 cDNA clones each (100-pools). Plasmides were extracted from 2000 of such 100-pools. The cDNA pools were cotransfected with each of the HLA-cDNAs of the respective model system in COS-7 and/or 293 T-cells and the transfectants were tested in the IFN-γ-ELISPOT assays for recognition by the T-cells. From positive 100-pools antigen-coding cDNAs were cloned. The inserts of the clones were sequenced. For comparison sequences from databases were used, as well as sequences of homologous cDNA from autologous EBV-B-transformed B-cells of both patients generated by means of RT-PCR.

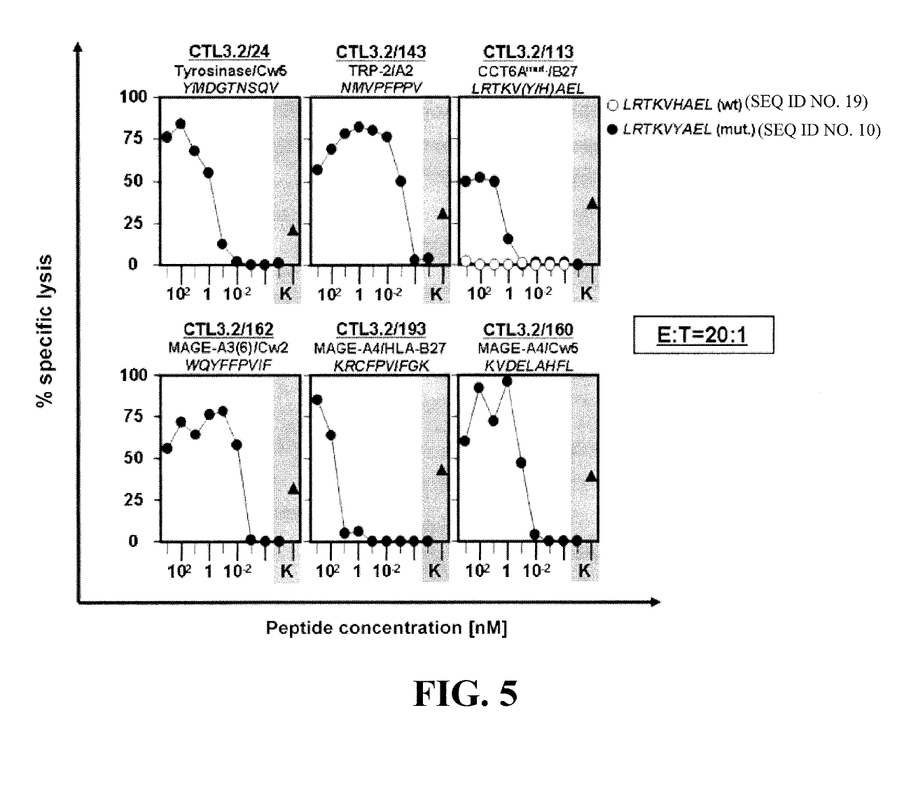

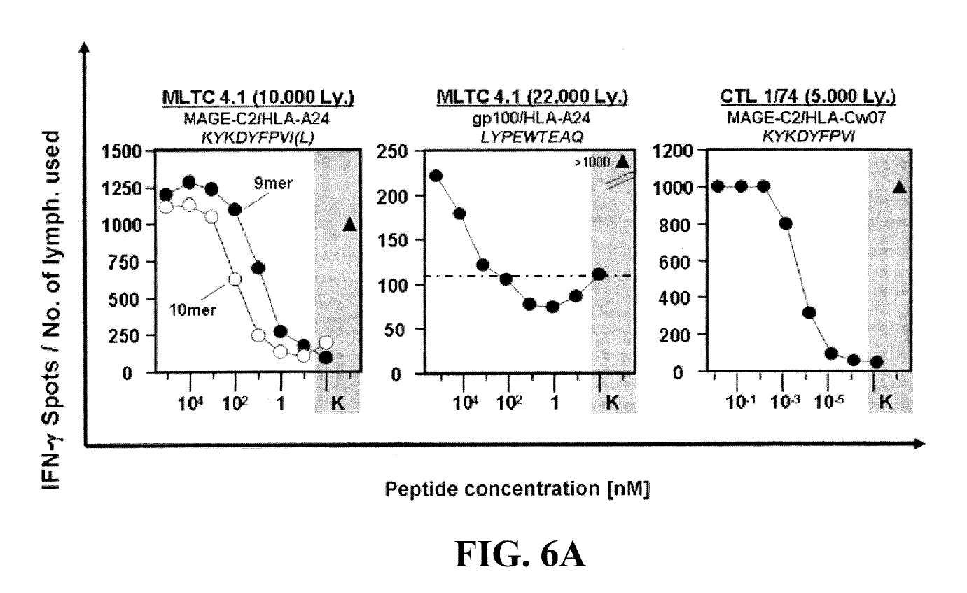

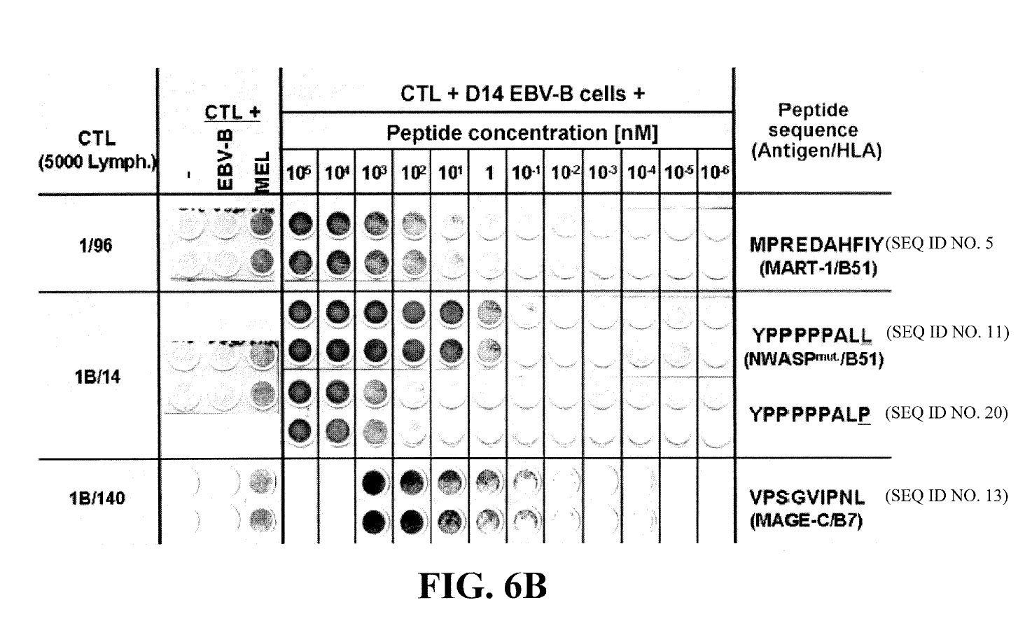

In two antigens (N-WASP in model D14-SJR and CCT6A (TCP20 is a synonym for CCT6A) in model D05) tumor-specific point mutations were found. Synthetic peptides, containing these mutations and binding to the presenting HLA-alleles according to available peptide algorithms, were synthesized and checked for T-cell recognition. In both cases the mutated peptides were recognized but not the homologous non-mutated peptide (CCT6A, FIG. 5), and the wild-type peptide was recognized 1000-times more poorly than the mutated one (N-WASP, FIG. 6B). Thus, the tumor-specific neomutations generated the immunogenic peptides. So far it is not known whether other melanomas also exhibit mutations of N-WASP and CCT6A. Another antigen found during bank screening, TRP-2, corresponds to a structurally normal melanosomal differentiation antigen that is also known as T-cell antigen. Via cDNA fragmentation the inventors found a novel peptide that is presented by HLA-A2, the most common HLA class I-allele in the Caucasian population (see Table 1).

FIG. 5 summarizes the recognition of all antigens from patient model D05-GS in so-called chromium release tests. With radioactive ⁵¹Cr chromated B-cells of the patient were titrated down with the indicated peptides in concentrations of 10³ nmol/1 to 10⁻⁴ nmol/l, loaded and coincubated with peptide-reactive T-cell clones for 4 hours. The recognition of the peptides by the T-cells resulted in the lysis of the B-cells and thereby release of the chromium from the cells, which was determined from the cell supernatant. (K in the grey area contains controls; the closed circle indicates recognition of the B-cells without peptide; the triangle shows recognition of autologous, chromated melanoma cells; E:T=ratio of effector cell to target cell).

FIGS. 6A and 6B summarize the recognition of the antigens from the patient model D14-SJR in IFN-γ ELISPOT assays. Here autologous B-cells or COS-7 or 293 T-cells transfected with individual HLA-cDNAs also served as antigen-presenting cells. These were loaded with the indicated peptides and coincubated with T-cells for 20 hours. In these tests the peptides were also titrated (K in the grey area in 6A contains controls: the close circle shows recognition of the B-cells without peptide; the triangle shows the recognition of the D14 melanoma cells.)

### LITERATURE

T. Wölfel, A. Van Pel, E. De Plaen, C. Lurquin, J. Maryanski and T. Boon. Immunogenic (tum⁻) variants obtained by mutagenesis of mouse mastocytoma P815. VIII. Detection of stable transfectants expressing a tum⁻ antigen with a cytolytic T cell stimulation assay. Immunogenetics 26: 178-187, 1987.
T. Wölfel, E. Klehmann, C. Müller, K.-H. Schütt, K.-H. Meyer zum Büschenfelde and A. Knuth. Lysis of human melanoma cells by autologous cytolytic T cell (CTL) clones: Identification of HLA-A2 as a restriction element for three different antigens. J. Exp. Med. 170:797-810, 1989.
V. Brichard, A. Van Pel, T. Wölfel, C. Wölfel, E. de Plaen, B. Lethé, P. Coulie, and T. Boon. The tyrosinase gene codes for an antigen recognized by autologous cytolytic T lymphocytes on HLA-A2 melanomas. J. Exp. Med. 178: 489-495, 1993.
T. Wölfel, A. Van Pel, V. Brichard, J. Schneider, B. Seliger, K.-H. Meyer zum Büschenfelde, and T. Boon. Two tyrosinase nonapeptides recognized on HLA-A2 melanomas by autologous cytolytic T lymphocytes. Eur. J. Immunol. 24:759-764, 1994.
P. Coulie, V. Brichard, A. Van Pel, T. Wolfel, J. Schneider, C. Traversari, S. Mattei, E. De Plaen, C. Lurquin, J.-P. Szikora, J.-C. Renauld, and T. Boon. A new gene coding for a differentiation antigen recognized by autologous cytolytic T lymphocytes on HLA-A2 melanomas. J. Exp. Med. 180:35-42, 1994.
T. Wölfel, M. Hauer, J. Schneider, M. Serrano, C. Wolfel, E. Klehmann-Hieb, E. De Plaen, T. Hankeln, K.-H. Meyer zum Büschenfelde, and D. Beach. A p16^{INK4a}-insensitive CDK4 mutant targeted by cytolytic T lymphocytes in a human melanoma. Science 269:1281-1284, 1995.
V. G. Brichard, J. Herman, A. Van Pel, C. Wildman, B. Gaugler, T. Wölfel, T. Boon, and B. Lethé. A tyrosinase peptide presented by HLA-B44 is recognized by autologous cytolytic T lymphocytes. Eur. J. Immunol. 26:224-230, 1996.
W. Herr, J. Schneider, A. W. Lohse, K.-H. Meyer zum Büschenfelde, and T. Wölfel. Detection and quantification of blood-derived T lymphocytes secreting tumor necrosis factor alpha in response to HLA-A2.1-binding melanoma and viral peptide antigens. J. Immunol. Methods 191:131-142, 1996.
W. Herr, B. Linn, N. Leister, E. Wandel, K.-H. Meyer zum Büschenfelde, and T. Wölfel. The use of computer-assisted video image analysis for the quantification of CD8⁺T lymphocytes producing tumor necrosis factor a spots in response to peptide antigens. J. Immunol. Methods 203:141-152, 1997.
J. Schneider, V. Brichard, T. Boon, K.-H. Meyer zum Büschenfelde, and T. Wölfel. Overlapping peptides of melanocyte differentiation antigen Melan-A/MART-1 recognized byautologous cytolytic T lymphocytes in association with HLA-B45.1 and HLA-A2.1. Int. J. Cancer 75:451-458, 1998.
S. Morel, A. Ooms, A. Van Pel, T. Wölfel, V. Brichard, P. van der Bruggen, B. Van den Eynde and G. Degiovanni. A new tyrosinase peptide presented by HLA-B35 is recognized on a human melanoma by autologous cytotoxic T lymphocytes. Int. J. Cancer 83:755-759, 1999.
C. Wölfel, I. Drexler, A. Van Pel, T. Thres, N. Leister, W. Herr, G. Sutter, C. Huber and T. Wölfel. Transporter (TAP)- and proteasome-independent presentation of a melanoma-associated tyrosinase epitope. Int. J. Cancer 88:432-438, 2000.
C. M. Britten, R. G. Meyer, T. Kreer, I. Drexler, T. Wölfel, W. Herr. The use of HLA-A*0201-transfected K562 as standard antigen-presenting cells for CD8(+) T lymphocytes in IFN-gamma ELISPOT assays. J. Immunol. Methods 259:95-110, 2002.
R. Konopitzky, U. König, R. G. Meyer, W. Sommergruber, T. Wölfel, and T. Schweighoffer. Identification of HLA-A*0201-restricted T cell epitopes derived from the novel overexpressed tumor antigen CLCA2. J. Immunol., 169:540-547, 2002.
C. M. Britten, R. G. Meyer, N. Frankenberg, C. Huber. T. Wölfel. The use of clonal mRNA as antigenic format for the detection of antigen-specific T lymphocytes in IFN-gamma ELISPOT assays. J. Immunol. Methods, 287:125-136, 2004.
A. Dörrschuck, A. Schmidt, E. Schnürer, M. Glückmann, C. Albrecht, C. Wölfel, V. Lennerz, A. Lifke, C. Di Natale, E. Ranieri, L. Gesualdo, C. Huber, M. Karas, T. Wölfel, and W. Herr. CD8+ cytotoxic T lymphocytes isolated from allogeneic healthy donors recognize HLA-class Ia/Ib-associated renal carcinoma antigens with ubiquitous or restricted tissue expression. Blood, 104:2591-2599, 2004.
Y. Zhang. Z. Sun. H. Nicolay, R. G. Meyer, N. Renkvist, V. Stroobant, J. Corthals, J. Carrasco, A. M. Eggermont, M. Marchand, K. Thielemans, T. Wölfel, T. Boon, P. van der Bruggen. Monitoring of anti-vaccine CD4 T cell frequencies in melanoma patients vaccianted with a MAGE-3 protein. J. Immunol. 174:2404-2411, 2005.
R. G. Meyer, C. M. Britten, U. Siepmann, B. Petzold, T. A. Sagban, H. A. Lehr, B. Weigle, M. Schmitz, L. Mateo, B. Schmidt, H. Bernhard, T. Jakob, R. Hein, G. Schuler, B. Schuler-Thurner, S. N. Wagner, I. Drexler, G. Sutter, N. Arndtz, P. Chaplin, A. Enk, C. Huber, and T. Wölfel. A phase I vaccination study with tyrosinase in patients with stage II melanoma using recombinant modified vaccinia virus Ankara (MVA-hTyr). Cancer Immunol. Immunother. 54:453-467, 2005.
C. M. Britten, R. G. Meyer, C. Graf, C. Huber, T. Wölfel. Identification of T cell epitopes by the use of rapidly generated mRNA fragments. J. Immunol. Methods 299:165-175, 2005.

1-3. (canceled)

4. A method of treating a patient against melanoma, comprising administering to the patient a therapeutically effective amount of at least one of:
i) a melanoma-specific immunogen, which is a peptide with a length from 9 to about 15 residues and which comprises an amino acid sequence selected from SEQ ID NOs. 2, 3, 6, 7, 8, 9, 10, 11, 12, 1, 4 and 5, wherein the immunogen elicits a melanoma-specific HLA-restricted CTL response and wherein the peptide does not correspond to the full length sequence of the underlying tumor antigen;
ii) a retro-inverse peptide or pseudo-peptide, characterized in that it corresponds to a melanoma-specific immunogen as described in i) and which is designed of —NH—CO— bonds or other non-peptide bonds instead of —CO—NH-peptide bonds;
iii) a fusion protein, consisting of a melanoma-specific immunogen described in i) or a retro-inverse peptide or pseudo-peptide described in ii) wherein said fusion protein further comprises a heavy chain of the HLA molecule and a flexible linker and is designed in such a way that the immunogen or retro-inverse peptide or pseudo-peptide is capable of occupying the peptide binding groove of the HLA molecule;
iv) a T cell receptor that specifically reacts with an immunogen as described in i), ii), or iii);
v) a polynucleotide encoding an immunogen or receptor as described in i), ii), iii), or iv); or
vi) an antibody that specifically reacts with an immunogen or receptor as described in i), ii), iii), or iv);
optionally together with suitable additives and excipients, thereby achieving a therapeutic effect.

5. (canceled)

6. The method, according to claim 4, wherein said method comprises the administration of a melanoma-specific immunogen, which is a peptide with a length from 9 to about 15 residues and which comprises an amino acid sequence selected from SEQ ID NO: 2, 3, 6, 7, 8, 9, 10, 11, 12, 1, 4 and 5, wherein the immunogen elicits a melanoma-specific HLA-restricted CTL response and wherein the peptide does not correspond to the full length sequence of the underlying tumor antigen

7. The method, according to claim 6, wherein the immunogen has a length of 9 to 11 residues.

8. The method, according to claim 6, wherein the immunogen is identical to one of the peptides of SEQ ID NO: 2, 3, 6, 7, 8, 9, 10, 11, 12, 1, 4 and 5.

9. The method, according to claim 6, wherein the immunogen has an amino acid sequence derivable by amino acid substitution, deletion, insertion, addition, inversion and/or by chemical or physical modification of one or more amino acids thereof,
wherein said amino acid sequence is a functional equivalent to the amino acid sequence of one of the peptides of SEQ ID NOs:1 to 12,
wherein said immunogen is an epitope for CD8-positive CTLs and is capable of inducing an immune response of CD8-positive CTLs against tumor cells, and
wherein said immune response is restricted to human leukocyte-antigen of the molecule group of MHC class I, allele variant A or B.

10. The method, according to claim 4, wherein said method comprises the administration of a retro-inverse peptide or pseudo-peptide, characterized in that it corresponds to a melanoma-specific immunogen as described in i) and which is designed of —NH—CO— bonds or other non-peptide bonds instead of —CO—NH-peptide bonds.

11. The method, according to claim 10, wherein said method comprises the administration of a retro-inverse peptide or pseudo-peptide, characterized in that it corresponds to a melanoma-specific immunogen, which is a peptide with a length from 9 to about 15 residues and which comprises an amino acid sequence selected from SEQ ID NO: 2, 3, 6, 7, 8, 9, 10, 11, 12, 1, 4 and 5, wherein the immunogen elicits a melanoma-specific HLA-restricted CTL response and wherein the peptide does not correspond to the full length sequence of the underlying tumor antigen, and which is designed of —NH—CO— bonds or other non-peptide bonds instead of —CO—NH-peptide bonds.

12. The method, according to claim 4, wherein said method comprises the administration of a fusion protein, consisting of a melanoma-specific immunogen described in i) or a retro-inverse peptide or pseudo-peptide described in ii) wherein said fusion protein further comprises a heavy chain of the HLA molecule and a flexible linker and is designed in such a way that the immunogen or retro-inverse peptide or pseudo-peptide is capable of occupying the peptide binding groove of the HLA molecule.

13. The method, according to claim 12, wherein said method comprises the administration of a fusion protein, consisting of:
i) a melanoma-specific immunogen, which is a peptide with a length from 9 to about 15 residues and which comprises an amino acid sequence selected from SEQ ID NO: 2, 3, 6, 7, 8, 9, 10, 11, 12, 1, 4 and 5, wherein the immunogen elicits a melanoma-specific HLA-restricted CTL response and wherein the peptide does not correspond to the full length sequence of the underlying tumor antigen; or
ii) a retro-inverse peptide or pseudo-peptide, characterized in that it corresponds to a melanoma-specific immunogen as described in i), and which is designed of —NH—CO— bonds or other non-peptide bonds instead of —CO—NH-peptide bonds;
wherein said fusion protein further comprises a heavy chain of the HLA molecule and a flexible linker and is designed such that the immunogen or retro-inverse peptide or pseudo-peptide is capable of occupying the peptide binding groove of the HLA molecule.

14. The method, according to claim 4, wherein said method comprises the administration of a T cell receptor that specifically reacts with an immunogen as described in Part i), ii), or iii) of claim 4.

15. The method, according to claim 4, wherein said method comprises the administration of a polynucleotide, comprising a nucleotide sequence encoding an immunogen as described in Part i), ii), or iii) of claim 4, or which encodes a T cell receptor that specifically reacts with an immunogen as described in Part i), ii), or iii) of claim 4.

16. The method, according to claim 4, wherein said method comprises administering an antibody that specifically reacts with an immunogen or receptor as described in Part i), ii), iii), or iv) of claim 4.


SEQUENCE LISTING (25 sequences)

SEQ ID NO: 1 - protein, 9 aa, Homo sapiens
WQYFFPVIF

SEQ ID NO: 2 - protein, 10 aa, Homo sapiens
KRCFPVIFGK

SEQ ID NO: 3 - protein, 9 aa, Homo sapiens
KVDELAHFL

SEQ ID NO: 4 - protein, 9 aa, Homo sapiens
NMVPFFPPV

SEQ ID NO: 5 - protein, 10 aa, Homo sapiens
MPREDAHFIY

SEQ ID NO: 6 - protein, 10 aa, Homo sapiens
LYPEWTEAQR

SEQ ID NO: 7 - protein, 9 aa, Homo sapiens
KYKDYFPVI

SEQ ID NO: 8 - protein, 10 aa, Homo sapiens
ASSASSTLYL

SEQ ID NO: 9 - protein, 9 aa, Homo sapiens
SSASSTLYL

SEQ ID NO: 10 - protein, 9 aa, Homo sapiens
LRTKVYAEL

SEQ ID NO: 11 - protein, 9 aa, Homo sapiens
YPPPPPALL

SEQ ID NO: 12 - protein, 9 aa, Homo sapiens
YMDGTMSQV

SEQ ID NO: 13 - protein, 9 aa, Homo sapiens
VPSGVIPNL

SEQ ID NO: 14 - protein, 8 aa, Homo sapiens
LAHFLLRK

SEQ ID NO: 15 - protein, 7 aa, Homo sapiens
LAHFLLR

SEQ ID NO: 16 - protein, 6 aa, Homo sapiens
LAHFLL

SEQ ID NO: 17 - protein, 5 aa, Homo sapiens
LAHFL

SEQ ID NO: 18 - protein, 4 aa, Homo sapiens
LAHF

SEQ ID NO: 19 - protein, 9 aa, Homo sapiens
LRTKVHAEL

SEQ ID NO: 20 - protein, 9 aa, Homo sapiens
YPPPPPALP

SEQ ID NO: 21 - protein, 9 aa, Homo sapiens
YMDGTNSQV

SEQ ID NO: 22 - protein, 8 aa, Homo sapiens
NMVPFPPV

SEQ ID NO: 23 - protein, 9 aa, Homo sapiens
  MISC_FEATURE (6)..(6): Xaa can be either Tyr or His
LRTKVXAEL

SEQ ID NO: 24 - protein, 10 aa, Homo sapiens
KYKDYFPVIL

SEQ ID NO: 25 - protein, 9 aa, Homo sapiens
LYPEWTEAQ